(19) United States
(12) Patent Application Publication
(10) Pub. No.: US 2013/0052113 A1
(43) Pub. Date: Feb. 28, 2013

(54) METHOD AND DEVICE FOR THE PURIFICATION OF THE AIR

(71) Applicants: Laurent Molins, Souppes Sur Long (FR); Audrey Durand, Souppes Sur Long (FR)

(72) Inventors: Laurent Molins, Souppes Sur Long (FR); Audrey Durand, Souppes Sur Long (FR)

(73) Assignee: SAINT-GOBAIN QUARTZ S.A.S., Courbevoie (FR)

(21) Appl. No.: 13/696,692

(22) PCT Filed: May 24, 2011
(86) PCT No.: PCT/FR2011/051170

§ 371 Date: Nov. 7, 2012

(30) Foreign Application Priority Data
May 25, 2010 (FR) 10 54045

(51) Int. Cl. A61L 9/20

(52) U.S. Cl. 423/245.1; 423/210; 422/121

(57) ABSTRACT

The invention relates to a method and a device for the purification of air by passing a flow of the air through a filter having a photocatalytic action subjected to UV lighting, said filter comprising a felt of mineral fibers, the fibers of which are coated with a material having a photocatalytic action, the residence time of the air in contact with the filter being greater than 70 msec and the UV lighting having a power of less than 35 mW per cm² of lit surface of filter having a photocatalytic action. The invention is particularly effective in removing volatile organic compounds from the air.

The invention relates to a method and to a device for purifying the air by freeing it from volatile organic compounds (VOCs). The device (also known as purifier) comprises a filtering media (or filter) having a fibrous structure, the fibers of which are coated with a coating having a photocatalytic action, and comprises ultraviolet lighting which activates said coating.

“Advanced Oxidation” techniques (AOTs) make it possible to oxidize volatile organic compounds (VOCs). The most effective are those which result in the formation of hydroxyl radicals OH., which have a greater oxidizing power than that of conventional oxidizing agents. This is the case with heterogeneous photocatalysis. Its principle is based on the absorption of a photon by a semiconducting solid, resulting in the promotion of an electron from the valence band to the conduction band with the release of a hole and thus conferring, on the solid, properties of oxidizing agent and of reducing agent. The majority of volatile organic compounds and also numerous pesticides, herbicides, surfactants and colorants are completely oxidized by this technique to give less toxic products.

The main VOCs in ambient air are: formaldehyde, acetaldehyde, acetone, octane, alcohols, including isopropanol, decane, benzene and toluene.

A PCO (photocatalytic oxidation) reactor for the purification of ambient air generally comprises a prefilter for trapping dust and particles, a UV source and a filter having a photocatalytic action (“PCO” filter). The UV source is generally placed between the prefilter and the PCO filter. The air to be purified is generally pulsed or sucked through the PCO filter using a turbine or a fan.

In air treatment applications, the design of the various components, fans, sheathing, engine power, is directly related to the pressure drop, which depends on the various filtration components of the system, including the PCO media.

The filters already provided for this type of application often cause an excessively large pressure drop, so that they require the use of fans which are more powerful, noisier and consume more energy. In order to overcome this disadvantage, provision was then made to lower the density of the filter by insertion of components, such as honeycomb, cloth with a high degree of porosity, mosquito screen or ceramic foam, but then true preferential channels are created and the efficiency of the filter for the oxidation of volatile organic compounds was thereby reduced as a result of the small amount of “effective” material in contact with the air stream.

WO 03/010106 teaches the deposition of photocatalytic coating at the surface of silica veils or felts with a specific surface at least equal to 10 m²/g, in particular at least equal to 30 m²/g.

WO 2009/019387 teaches a filtering media having a photocatalytic action which has a thickness of at least 2 mm, which is homogeneous and which is devoid of orifice apparent to the naked eye, comprising a felt of inorganic fibers, the fibers of which are coated with a coating comprising a catalyst having a photocatalytic action, said felt exhibiting a weight per unit area of between 30 and 80 g/m², said coating representing from 5 to 80% of the weight of said media, said media exhibiting a gas pressure drop of less than 150 Pa at 1 m/s in unpleated condition.

WO 2009/019388 teaches a gas purifier comprising a filtering media having a photocatalytic action, a system for illuminating said media with UV radiation, a time-delay means or an analyzer of volatile organic compounds, a means for automatically adjusting the speed of the gas passing through it or for adjusting the intensity of the UV illumination, said adjusting being carried out as a function of the time determined by the time-delay means or as a function of the content of a volatile organic compound analyzed by the analyzer. This document also describes a method for the purification of gas using a purifier comprising a filtering media having a photocatalytic action and a system for illuminating said media with UV radiation, according to which, when the concentration of a compound in the gas is greater than a value V1, the operating rate of the purifier is lower than its rate when the concentration of the compound in the gas is lower than a value V2, V2 being less or equal to V1. This document also describes a method for the purification of gas using a purifier comprising a filtering media having a photocatalytic action, a system for illuminating said media with UV radiation and a time-delay means which controls the rate of the purifier.

Mention may also be made, as documents of the prior art, of U.S. Pat. No. 4,732,879 A1. This document teaches the deposition of a porous catalytic coating on a flexible fibrous substrate composed of glass or ceramic fibers.

The decomposition of VOCs by a PCO filter can result in the formation of by-products also of the VOC type. In point of fact, it is found that purifiers frequently discharge at least one of the following impurities: formaldehyde, acetaldehyde or acetone. Specifically, as an example of a series of chemical decomposition reactions in a purifier, a process for the decomposition of methanol is: Methanol→Formaldehyde→Formic acid→CO₂. As an example of a series of chemical reactions, a process for the decomposition of ethanol is: Ethanol→Acetaldehyde→Acetic acid→Formaldehyde→Formic acid+CO₂→2 CO₂. The intermediate reaction products may be completely or partially converted to inorganic compounds during the passage of the gas to be purified through the photocatalytic media.

It is difficult to understand that a purifier discharges VOCs when it is supposed to purify the air, even if these discharged VOCs are different in nature and/or in amount from what was present in the starting air.

In order to reduce the formation of these toxic by-products, WO 2009/019388 provided for the lowering of the operating rate (speed of the gases or UV intensity) of the purifier until the VOC content of the air to be purified falls below a certain value. Even if this solution works, it is regrettable not to be able to operate the purifier precisely when it is most needed, that is to say in the presence of a high concentration of VOCs.

The invention solves the abovementioned problems. This is because it has been found that the essential parameter not only for efficiently reducing the starting VOC content but also for preventing the formation of VOCs by the purifier itself was the residence time of the gas in contact with the media having a photocatalytic action (also known here as “PCO media”). It has been determined that this residence time should be greater than 70 milliseconds and that the UV lighting should have a power of less than 35 mW per cm² of lit surface of filter having a photocatalytic action. The filter surface concerned is that actually lit. By virtue of a purifier designed so that the residence time in contact with the PCO media is at least 70 milliseconds (msec), and moderate UV lighting, the concentrations of formaldehyde and acetaldehyde are drastically reduced.

The residence time RT is calculated from the surface area S of the filter, from the thickness T of the filter and from the gas flow rate GF according to: RT=(S·T)/GF

The surface area S is the macroscopic surface area of the filter. Thus, if the filter has a parallepipedal shape, S.E represents its bulk volume.

The residence time can be increased by increasing the surface area of the PCO media, by increasing its thickness or by reducing the flow rate. The reduction in the flow rate is not the best solution insofar as it is desired to treat as quickly as possible the largest volume of gas possible. In particular, it would not be acceptable to operate with hourly flow rates which are less than one times the volume of the space to be treated. The flow rate in the purifier is, in one hour, preferably at least 2.5 times and preferably at least 3 times the volume of the space to be treated.

Surprisingly, the inventors have been able to find that the level of formaldehyde exiting from a purifier passes through a maximum when the residence time is increased. This is due to the fact that the purifier itself tends to generate formaldehyde when the incoming air comprises VOCs other than formaldehyde, in particular an alcohol or an alkene. This behavior is confirmed whatever the density of the filter and the true fiber surface area in the filtering media. Thus, the invention also relates to a method for the purification of air comprising an alcohol or an alkene.

According to the invention, a residence time is imposed on the gas to be purified such that this maximum is exceeded. This residence time is greater than 70 msec, preferably greater than 80 msec and more preferably greater than 100 msec.

Use is made, as support of the PCO media, of a structure made of mineral fibers, in particular of pure silica or of glass or of metal or of ceramic. The PCO media comprises a fibrous structure, the fibers of which are coated with a material having a photocatalytic action. Preferably, the fibrous structure is a felt comprising mineral fibers. In a felt, the fibers are entangled and do not all have the same direction. Preferably, the felt is devoid of particles between the fibers as particles are capable of generating gaps forming preferential channels for the gas to be treated, which can lower the efficiency of the media. Remarkably, the media according to the invention is very effective without it being necessary to provide a very great thickness of PCO filtering media in the direction of the gas stream to be purified. Specifically, the filter according to the invention can have a thickness of less than 50 mm. It generally has a thickness of greater than 3 mm. In particular, it can have a thickness of between 5 and 25 mm. Even with such a lack of thickness, it is possible to purify gas flow rates of greater than 10 m³/h, in particular of greater than 50 m³/h, such as from 50 to 500 m³/h.

The mineral fibers preferably have a diameter of less than 40 μm (their cross section comes within a circle with a diameter of less than 40 μm). Their diameter is generally between 0.5 and 30 μm, in particular between 5 and 20 μm. The support felt preferably exhibits, by mercury porosimetry, a mean pore diameter of between 50 and 1000 μm and a degree of porosity of between 30 and 95% (volume between the fibers).

The mineral fibers can be silica-based, such as glass (generally comprising at least 30% by weight of silica, it being possible for the glass to be of the E, C, R, S, D or AR type), washed glass (glass fiber leached chemically and then possibly stabilized thermally, generally comprising more than 90% by weight of silica and, in a standard way, between 96% and 99% by weight of silica), made of ceramic (mention may be made of mullite-based fibers, of which Unifrax and Thermal Ceramics are suppliers, Nextel fibers from 3M or the pure alumina fiber sold under the Saffil tradename), or pure silica (also known as quartz and comprising at least 99% of amorphous SiO₂). A felt of fibers of pure molten silica, in particular of the Quartzel trademark (registered trademark of Saint-Gobain Quartz SAS), is particularly suitable. The Quartzel felt has the following characteristics:
diameter of the mineral fibers: mean diameter of 10 μm, all the fibers having a diameter of between 0.5 and 30 μm
mean pore diameter: 220 μm
degree of porosity (mercury): 85%.

Reference may in particular be made to WO 2009/019387 for the preparation of a PCO media.

The material having a photocatalytic action generally comprises at least one oxide from the group of the following oxides: TiO₂, ZnO and CeO₂. It preferably comprises at least partially crystalline titanium oxide.

The PCO media is illuminated with UV radiation in order for the PCO coating to properly act with regard to the VOCs. The UV intensity is less than 35 mW per cm² of lit PCO media and more preferably less than 25 mW per cm² of lit PCO media. This is because an excessively strong UV intensity excessively strongly activates the decomposition of the VOCs by the filter having a photocatalytic action, which is reflected by the generation of excessively high amounts of other VOCs by the filter itself. The intensity of the UV lighting is preferably greater than 1 mW per cm² and more preferably greater than 5 mW per cm² of lit surface of filter having a photocatalystic action (PCO media). Use may be made, as UV radiation, of UV-A radiation or UV-C radiation. UV-C radiation is sometimes chosen because of its germicidal power. On the other hand, it is regarded as being more dangerous to the eyes. The choice between UV-A and UV-C radiation depends on the uses and regulations according to country.

For the case where geometrical constraints dictate the emergence, despite everything, of VOCs downstream of the PCO media, it is possible to position, in the pathway of the air, another media, referred to as scavenger media, the role of which is to at least partially retain the VOCs, if appropriate after chemical reaction. The various categories of scavenger media are thus distinguished:
those which retain the VOCs, such as formaldehyde or acetaldehyde, by simple physical interaction, as is the case with active charcoal or some zeolites,
those which interact chemically with the VOCs, such as formaldehyde or acetaldehyde, and allow the escape of the carbon-based molecules resulting from the reaction of the VOC, such as formaldehyde or acetaldehyde, with the scavenger media: this is the case with chemical filters comprising a permanganate, such as potassium permanganate,
those which interact chemically with the VOCs, such as formaldehyde or acetaldehyde, without allowing the escape of carbon-based molecules originating from the reaction of said VOC, such as formaldehyde or acetaldehyde, with the scavenger media: this is the case with scavenger medias comprising a compound comprising an NH group; the chemical reaction is in this instance rather of the grafting type since the reaction between the N—H group (substance on the scavenger media) and the C═O group of the formaldehyde or acetaldehyde results in a sequence of the N—C—OH type without formation of a volatile carbon-based molecule.

The term “scavenger” is thus understood to mean that the scavenger media at least partially retains the formaldehyde or the acetaldehyde, either by a chemical reaction (for example a grafting reaction) or by a physical interaction of the resorption or absorption type.

It is also possible, in order to improve the efficiency of the purifier, to place a scavenger media upstream of the filter having a photocatalytic action in the case of a purifier operating with recirculation, that is to say causing the air from the room to be purified to go round in a loop (open or semi-open loop). In this operating form, this VOC-scavenging media can also be used as a dust filter. In all the cases, a particle filter (also known as prefilter) is generally placed in the very first position on the route of the gas to be purified, which necessarily results in a certain pressure drop. The fact of using the scavenger media also as a particle filter contributes to reducing the pressure drops caused since just one filter acts as two filters. A particle filter is characterized by its arrestance, which determines its classification into one of the classes G1 to G4, F5 to F9, H10 to H14 and U15 to U17.

Thus, the invention also relates to a purifier comprising a filtering media having a photocatalytic action (“PCO” media) distinct from the media comprising the scavenger, and UV lighting in order to activate said PCO media.

It is also possible to prepare a media which is both a scavenger media and a media having a photocatalytic action. To do this, in the formula for the preparation of the media by deposition of a material having a photocatalytic action at the surface of a support, such as a fibrous support, for example, scavenger material (or compound), preferably of the absorber type, such as a zeolite, is added to the preparation forming a material having a photocatalytic action.

The scavenging media comprises, as support for the VOC-scavenging compound, a solid structure, such as, for example, a structure made of mineral fibers, in particular of silica or of glass or of metal or of ceramic, or a honeycomb structure made of metal or of ceramic or of glass or of plaster, or a foam structure made of metal or of ceramic or of glass or of plaster.

Mention may be made, as scavenger compound (or material) of:
diesters (e.g.: ethylene bis(acetoacetate) and oxydiethylene bis(acetoacetate)),
β-dicarbonyl compounds (e.g.: acetoacetamide, acetaoacetone, ethyl acetoacetate, and the like),
polyols (e.g.: ethylene glycol),
amides (e.g.: urea, ethylene urea, and the like),
hydroxylamines (e.g.: benzylhydroxylamine),
amines (e.g.: polyvinylamine, arginine, lysine, and the like),
polyalkyleneimines (e.g.: polyethyleneimine),
acetals (e.g.: diethylene glycols, saccharides, and the like),
phenolic compounds (e.g.: resorcinol),
sulfur-comprising compounds (e.g.: sodium bisulfite, cysteine, and the like),
aminoplasts (melamine/urea/formaldehyde resin).

Use may also be made, as scavenger material, of an absorbent, such as, for example, a hydrophobic silica zeolite, commonly known as “Zeosil”, such as that of the Sicade-1 trademark sold by Zéphir Alsace.

Use may also be made, as scavenger material, of active charcoal or potassium permanganate. However, active charcoal exhibits a relatively low efficiency. Potassium permanganate exhibits the disadvantage of reacting with the formaldehyde or the acetaldehyde, releasing other VOCs into the atmosphere. Use is made, as preferred scavenger media, of a media comprising a compound comprising an NH group. This is because this type of compound can react with the formaldehyde or the acetaldehyde without releasing carbon-based molecules, which is particularly advantageous in a context of protecting the environment. Acetoacetamide is a preferred scavenger compound.

Use is generally made of from 5 to 3000 g of scavenger compound per m² of support felt of fibrous structure. This fibrous structure can be chosen from the same materials as for the fibrous support of the PCO media. Preferably, use is made of a mineral fibrous material, in particular a felt formed of fibers of pure molten silica, in particular of the Quartzel trademark (registered trademark of Saint-Gobain SAS). In order to attach the scavenger compound to the fibers, an impregnation is carried out by dipping the carrier fibrous support in a solution or suspension of the scavenger material or by spraying a solution or suspension of the scavenger compound over the carrier fibrous support.

Preferably, the scavenger media brings about a pressure drop of less than 100 Pa at 2 m/s on the air passing through it.

The scavenger media is generally placed behind the PCO media (that is to say, downstream of the filter having a photocatalytic action) if the direction of the gas passing through the purifier is considered. It can be placed side by side with the PCO media or can be separated from it, for example by 5 to 60 cm (free space between the medias).

If a UV-C source is used in the purifier to excite the media having a photocatalytic action and if a scavenger media is also incorporated in the purifier, then the scavenger media and the UV-C source are preferably positioned so that the scavenger media is illuminated as little as possible by the UV-C source.

As the scavenger media retains the PCO molecules, if appropriate while converting them (chemical grafting or other), it accumulates them over time and has to be replaced or regenerated when it is saturated. In the case of the purification of indoor air (air of domestic premises, in contrast to “industrial air”), the scavenger media can be replaced or regenerated of the order of once annually. This maintenance with regard to the scavenger media is not in fact too restricting since the purifier has in any case to be maintained by the regular replacement (of the order of once annually) of the UV lighting. A scavenger media operating on the principle of the physical interaction (absorption or adsorption) with the VOCs can be regenerated by heat treatment. The heat treatment is sufficient to desorb the molecules retained on the scavenger media. Of course, it is also possible very simply to replace it. For the scavenger medias operating on the principle of a chemical reaction with the VOCs, it is necessary to replace them when they are saturated.

The purifier according to the invention relates to the purification of a standard indoor air (air of domestic premises, in contrast to “industrial air”) and that of an industrial air. In an indoor air, aldehydes (in particular formaldehyde and acetaldehyde) are among the VOC molecules which are the most frequent and the most concentrated in dwellings (observed in 99.4 to 100% of dwellings, according to the compounds). In addition, hydrocarbons are frequent therein (detected in 83 to 100% of dwellings, according to the compounds). Glycol ethers are relatively infrequent therein (detection in 2.3 to 85% of dwellings, according to the compounds). Biological contaminants (such as allergens from cats, dogs and mites) are present in significant amounts in 50% of dwellings. These data are described in the document “Observatoire de la qualité de l'air intérieur: Campagne Nationale Logements: Etat de la qualité d l'air dans les logements français Rapport final” [French Indoor Air Quality Observatory: National Dwellings Campaign State of the quality of the air in French dwellings—Final report]—Ref: DDD/SB-2006-57, November 2006—Séverine Kirchner et al.

In the case of the air of industrial premises, the concentrations of contaminants may be much higher, according to the type of industry. In the composites industry, styrene is a major contaminant of the air. The levels of by-products may also be high.

The invention also relates to an air purifier configured in order to employ the method according to the invention. This purifier comprises a means which forces the air to pass through it (fan or turbine) and which is configured in order to ensure that the residence time of the air in contact with the filter is greater than 70 msec. It also comprises UV lighting with a power of less than 35 mW per cm² and generally of greater than 1 mW per cm² of lit filter having a photocatalytic action. The purifier can in particular be incorporated in a centralized air conditioning system (for several residences) referred to as HVAC (heating, ventilation and air-conditioning).

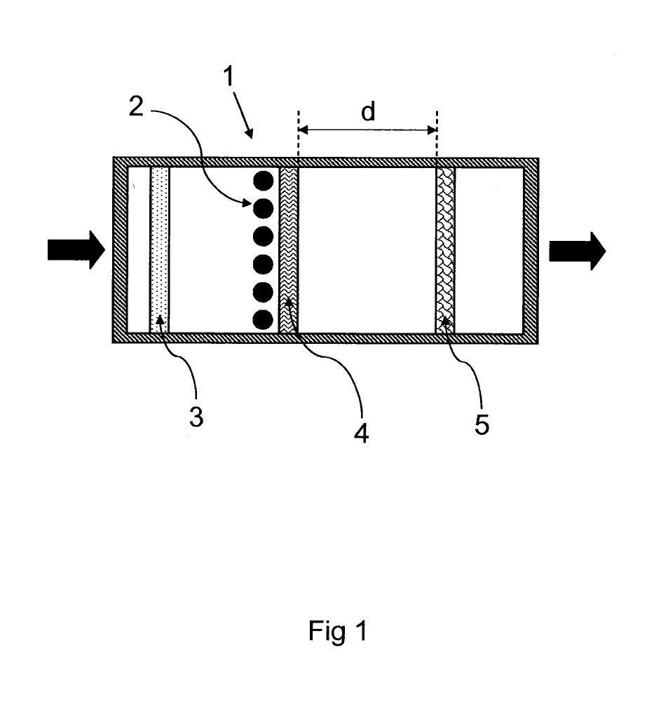

FIG. 1 represents a purifier 1 according to the invention through which air passes in the direction indicated by the arrows. It comprises UV lighting 2, a prefilter 3 for halting the particles, a PCO media 4 and a scavenger media separated from the PCO media by a distance d (free space between the medias).

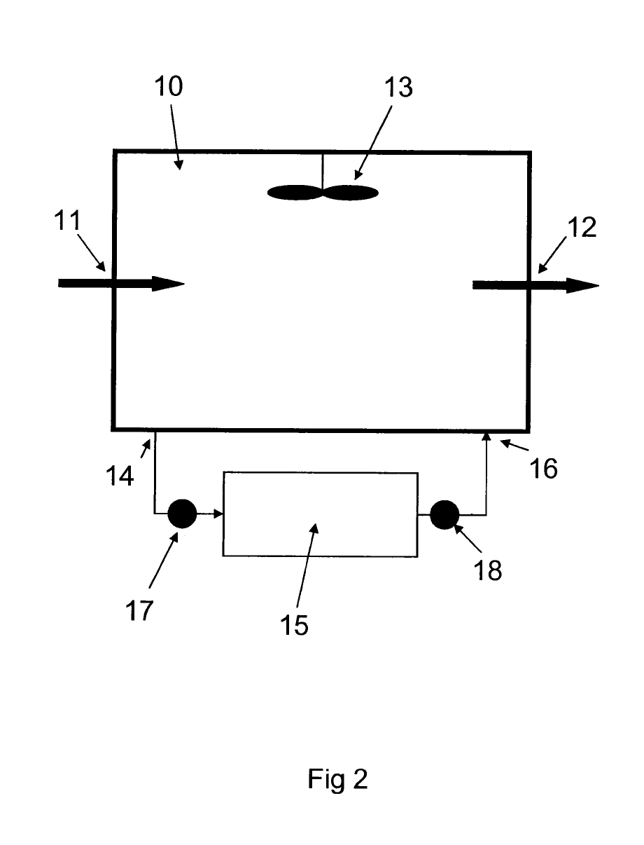

FIG. 2 represents a device used to carry out the measurements of the examples. A chamber 10 with a capacity of 20 m³ is fed continuously with contaminated air entering at 11. An equivalent volume of air is extracted at 12. The air of the chamber is homogenized with a fan 13. An air circulation loop is connected externally to this chamber, from which it withdraws air at 14. This air passes through a purifier 15 and is discharged into the chamber 10 at 16. Air is withdrawn for analysis at 17 and at 18, that is to say respectively before and after passing the air through the purifier, in order to evaluate its performance.

### EXAMPLES 1 TO 4

A purifier is prepared comprising, in the order of the passage of the air to be purified, UV lighting and a PCO media. The purifier is placed in a loop which makes possible the treatment of air withdrawn from a test chamber with a capacity of 20 m³, as represented in FIG. 2. The UV lighting is composed of UV-A or UV-C lamps (according to the examples) with a power of 40 W. The PCO media itself was prepared according to example No. 33 of WO 2009/019387. The area of its main geometrical surface (orthogonal to the direction of the air flow) was 0.3 m² and its thickness was 10 mm. The UV power was thus 13.3 mW/cm².

The implementational conditions and results of the examples are combined in table 1. The VOCs introduced in the air to be purified were a mixture composed of o-xylene, undecane, benzene, 1-butanol, toluene, formaldehyde and acetaldehyde. At the outlet of the system, the contaminants are captured on a Tenax tube (porous polymeric resin which makes it possible to trap VOCs). The Tenax tubes are subsequently analyzed by thermal desorption/gas chromatography coupled to a mass spectrometer. The aldehydes and ketones are captured on DNPH (2,4-dinotrophenylhydrazine) and then analyzed by HPLC (liquid chromatography).

TABLE 1

| | | | | | | % Variation |
|---|---|---|---|---|---|---|
| | | | | | | in VOCs |
| | | Linear | | | | excluding |
| | | velocity | | % Variation | % Variation | formaldehyde |
| Ex. | UV | of the air | Residence | in | in | and |
| No. | type | stream | time (ms) | formaldehyde | acetaldehyde | acetaldehyde |
| 1 | UV-C | 0.95 | 27 | +100% | −20% | −48% |
| 2 | UV-C | 0.39 | 65 | +50% | −30% | −60% |
| 3 | UV-C | 0.17 | 155 | −18% | −49% | −65% |
| 4 | UV-A | 0.17 | 155 | −24% | −35% | −71% |

The “% Variation” columns in table 1 indicate the percentage of increase or decrease in VOCs on passing through the purifier. In terms of reduction in total VOCs, whatever the residence time, the balance is positive since a reduction in the VOCs excluding formaldehyde and acetaldehyde of 48 to 71% is found. The photocatalytic activity with regard to the total VOCs thus increases with the residence time. In terms of by-products (acetaldehyde and formaldehyde), a residence time of greater than 70 ms is necessary in order not to generate by-products. A residence time of 155 ms makes it possible to get rid of 18% of the formaldehyde under UV-C illumination.

### EXAMPLES 5 TO 8

The procedure is the same as for examples 1 to 4 except that a scavenger media was added in the final position on the path of the gases, as represented in FIG. 1. In order to produce this scavenger media, use is made, as scavenger material, of α-acetylacetamide (C₄H₇NO₂) (also known as acetoacetamide), manufactured by Lonza and sold by Sigma-Aldrich under the reference 688789. This acetoacetamide is provided in the powder form. It is dissolved in ethanol in a proportion of 100 g of acetoacetamide in 500 ml of ethanol. This liquid preparation is subsequently deposited on a felt of the Quartzel trademark with a weight per unit area of 80 g/m². The support is impregnated with the acetoacetamide solution and dried at ambient temperature, so that 250 g of acetoacetamide are deposited on 0.3 m² of fibrous support.

The test conditions and the results are collated in table 2.

TABLE 2

| | | | | | | % Variation |
|---|---|---|---|---|---|---|
| | | | | | | in VOCs |
| | | Linear | | | | excluding |
| | | velocity | | % Variation | % Variation | formaldehyde |
| Ex. | UV | of the air | Residence | in | in | and |
| No. | type | stream | time (ms) | formaldehyde | acetaldehyde | acetaldehyde |
| 5 | UV-C | 0.95 | 27 | 15% | −25% | −48% |
| 6 | UV-C | 0.39 | 65 | −15% | −35% | −60% |
| 7 | UV-C | 0.17 | 155 | −40% | −56% | −65% |
| 8 | UV-A | 0.17 | 155 | −55% | −65% | −71% |

It should be noted that the presence of a scavenger media postfilter has no effect on the level of reduction in the VOCs excluding formaldehyde and acetaldehyde. As in the preceding system (without scavenger media), the level of reduction in the total VOCs is between 45 and 71%, according to the residence time. The scavenger media makes it possible to scavenge the by-products of formaldehyde and acetaldehyde type. For the formaldehyde, a positive balance is obtained from 65 ms (against 155 ms for the system without a scavenger media). For the acetaldehyde, a positive balance is obtained from 27 ms with a reduction of 25% (against a reduction of 20% at 27 ms with the system without a scavenger media).

### EXAMPLE 9

The procedure is the same as for examples 5 to 8 using a 40 W UV-C lamp and except that the nature of the contaminant is modified. Air contaminated by 25 ppm of methanol is introduced into the purifier at the rate of 383 liters per minute. A concentration of methanol of 18 ppm is measured between the two media. A concentration of methanol of 18 ppm is measured after the scavenger media. A reduction of greater than 38% in the level of formaldehyde is observed between the upstream side and the downstream side of the scavenger media.

1. A method for purifying air, the method comprising:
passing a flow of air through a filter having a photocatalytic action subjected to UV lighting, the filter comprising a felt comprising a mineral fiber coated with a material having a photocatalytic action,
wherein the residence time of the air in contact with the filter is greater than 70 msec and wherein the UV lighting has a power of less than 35 mW per cm² of lit surface of filter having a photocatalytic action.

2. The method of claim 1, wherein the residence time of the air in contact with the filter is greater than 80 msec.

3. The method of claim 2, wherein the residence time of the air in contact with the filter is greater than 100 msec.

4. The method of claim 1, wherein the UV lighting has a power of greater than 1 mW per cm² of lit surface of filter having a photocatalytic action.

5. The method of claim 1, wherein the mineral felt comprises fibers having a diameter of less than 40 μm and exhibits, by mercury porosimetry, a mean pore diameter of between 50 and 1000 μm and a degree of porosity of between 30 and 95%.

6. The method of claim 1, further comprising:
passing the air through a VOC-scavenging media, optionally after a chemical reaction.

7. The method of claim 6, wherein the scavenger media comprises a compound comprising an NH group.

8. The method of claim 6, wherein the scavenger media is a hydrophobic silica zeolite.

9. The method of claim 6, wherein the scavenger media is placed downstream of the filter having a photocatalytic action.

10. The method of claim 6, wherein the scavenger media is placed upstream of the filter having a photocatalytic action.

11. The method of claim 10, wherein the scavenger media retains dust.

12. The method of claim 1, wherein the air to be purified comprises an alcohol or an alkene.

13. The method of claim 1, wherein the air to be purified is in a space, the flow rate of the air through the filter being, in one hour, at least 2.5 times the volume of the space to be treated.

14. The method of claim 1, wherein the flow rate of the air through the filter is greater than 10 m³/h.

15. An air purifier, comprising
a filter having a photocatalytic action, UV lighting of the filter having a photocatalytic action with a power of between 1 and 35 mW per cm² of filter having a photocatalytic action; and
a fan or turbine, which forces the air to pass through it and which is configured in order for the residence time of the air in contact with the filter to be greater than 70 msec.

16. The air purifier of claim 15, employing the method of claim 1.

17. The air purifier of claim 15, being incorporated in a centralized air conditioning system.
